(19) United States
(12) Patent Application Publication
(10) Pub. No.: US 2021/0312330 A1
(43) Pub. Date: Oct. 7, 2021

(54) METHOD FOR TRANSFER LEARNING IN CLUSTERING

(71) Applicant: KONINKLIJKE PHILIPS N.V., EINDHOVEN (NL)

(72) Inventor: Jan Johannes Gerardus DE VRIES, Leende (NL)

(21) Appl. No.: 17/220,006

(22) Filed: Apr. 1, 2021

Related U.S. Application Data
(60) Provisional application No. 63/005,598, filed on Apr. 6, 2020.

(51) Int. Cl. G06N 20/00; G06F 16/35; G06F 16/27; G16H 10/60

(52) U.S. Cl. CPC G06N 20/00; G16H 10/60; G06F 16/27; G06F 16/35

(57) ABSTRACT

A method for clustering patients based upon unlabeled patient medical data, including: receiving a first feature of interest from a first user; extracting first patient data from a first patient database based upon the first feature of interest; labeling the extracted first patient data based upon the first feature of interest; producing a first customized distance measure using a classifier on the labeled patient data; extracting first unlabeled patient data from a second patient database; clustering the first unlabeled patient data using a clustering technique and the first customized distance measure to produce first clustered results.

### CROSS-REFERENCE TO PRIOR APPLICATIONS

This application claims the benefit of U.S. Provisional Application No. 63/005,598, filed on 6 Apr. 2020. This application is hereby incorporated by reference herein.

### TECHNICAL FIELD

Various exemplary embodiments disclosed herein relate generally to a method for transfer learning in clustering that allows for the application of train clustering to new datasets.

### BACKGROUND

Large amounts of medical data is currently available for evaluation by doctors and medical administrators. Such data may be used to identify patients that are similar in some way by using clustering techniques. These identified groups may have common characteristics that provide beneficial information to doctors and medical administrators.

### SUMMARY

A summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

Various embodiments relate to a method for clustering patients based upon unlabeled patient medical data, including: receiving a first feature of interest from a first user; extracting first patient data from a first patient database based upon the first feature of interest; labeling the extracted first patient data based upon the first feature of interest; producing a first customized distance measure using a classifier on the labeled patient data; extracting first unlabeled patient data from a second patient database; clustering the first unlabeled patient data using a clustering technique and the first customized distance measure to produce first clustered results.

Various embodiments are described, wherein the second patient database is the same as the first patient database.

Various embodiments are described, further including: receiving a second feature of interest from a second user; extracting second patient data from a second patient database based upon the second feature of interest; labeling the extracted second patient data based upon the second feature of interest; producing a second customized distance measure using a classifier on the second labeled patient data; and clustering the second unlabeled patient data using a clustering technique and the second customized distance measure to produce second clustered results.

Various embodiments are described, wherein the feature of interest is a continuous value.

Various embodiments are described, wherein the feature of interest is categorical.

Various embodiments are described, wherein the feature of interest is a binary value.

Further various embodiments relate to a non-transitory machine-readable storage medium encoded with instructions for clustering patients based upon unlabeled patient medical data, including: instructions for receiving a first feature of interest from a first user; instructions for extracting first patient data from a first patient database based upon the first feature of interest; instructions for labeling the extracted first patient data based upon the first feature of interest; instructions for producing a first customized distance measure using a classifier on the labeled patient data; instructions for extracting first unlabeled patient data from a second patient database; instructions for clustering the first unlabeled patient data using a clustering technique and the first customized distance measure to produce first clustered results.

Various embodiments are described, wherein the second patient database is the same as the first patient database.

Various embodiments are described, further including: instructions for receiving a second feature of interest from a second user; instructions for extracting second patient data from a second patient database based upon the second feature of interest; instructions for labeling the extracted second patient data based upon the second feature of interest; instructions for producing a second customized distance measure using a classifier on the second labeled patient data; and instructions for clustering the second unlabeled patient data using a clustering technique and the second customized distance measure to produce second clustered results.

Various embodiments are described, wherein the feature of interest is a continuous value.

Various embodiments are described, wherein the feature of interest is categorical.

Various embodiments are described, wherein the feature of interest is a binary value.

Further various embodiments relate to a device, for clustering patients based upon unlabeled patient medical data including: a memory; a processor coupled to the memory, wherein the processor is further configured to: receive a first feature of interest from a first user; extract first patient data from a first patient database based upon the first feature of interest; label the extracted first patient data based upon the first feature of interest; producing a first customized distance measure using a classifier on the labeled patient data; extract first unlabeled patient data from a second patient database; cluster the first unlabeled patient data using a clustering technique and the first customized distance measure to produce first clustered results.

Various embodiments are described, wherein the second patient database is the same as the first patient database.

Various embodiments are described, wherein the process is further configured to: receive a second feature of interest from a second user; extract second patient data from a second patient database based upon the second feature of interest; label the extracted second patient data based upon the second feature of interest; produce a second customized distance measure using a classifier on the second labeled patient data; and cluster the second unlabeled patient data using a clustering technique and the second customized distance measure to produce second clustered results.

Various embodiments are described, wherein the feature of interest is a continuous value.

Various embodiments are described, wherein the feature of interest is categorical.

Various embodiments are described, wherein the feature of interest is a binary value.

### BRIEF DESCRIPTION OF THE DRAWINGS

In order to better understand various exemplary embodiments, reference is made to the accompanying drawings, wherein:

FIG. 1 illustrates a block diagram for a user defined transferable clustering system; and

FIG. 2 illustrates an exemplary hardware diagram 200 for implementing the user defined transferable clustering system of FIG. 1.

To facilitate understanding, identical reference numerals have been used to designate elements having substantially the same or similar structure and/or substantially the same or similar function.

### DETAILED DESCRIPTION

The description and drawings illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments.

In clustering (unsupervised learning), data is grouped according to a similarity measure. The end-result is that the data is divided into groups where samples in the same group are more similar than samples of different groups. This depends on a good measure of similarity. There are a wide variety of known clustering techniques that may be applied to unlabeled data.

From a data perspective, it is impossible to optimize the measure of similarity (in the context of clustering) on unlabeled data alone as there exist no ground truth, because the grouping occurs independent of any labels or desired grouping that may be identified by labels. In supervised learning, where the data has labels that can be used for grouping, such optimization is possible. In the embodiments described herein, use is made of labels to define a way of transferring results from supervised to unsupervised learning using a customized distance measure.

This enables layman-users to design similarity measures that direct the clustering to show results that correspond to their expectation. Typically end-users have some idea of what kind of separation they would like to observe, but don't know which features should be used for the similarity measure. For example, an administrator wants to group patients by total overall cost or by the likelihood of readmission. A doctor may want to group patients by the likelihood that a current medical condition is going to get worse. Yet, choosing the correct similarity measure is key to obtaining valuable clustering results. The embodiments described herein provides an automated way of choosing an appropriate distance measure given a wished-for separation. This method allows for easy transfer of a once-calibrated-distance-measure to a new dataset. This method provides a means to transfer knowledge from the application of supervised learning to unsupervised learning and by doing so, direct clustering towards showing separation in terms of properties an end-user would expect or like to see. Also, these embodiments allows for reusing the distance measure when clustering is applied repeatedly over time to new unlabeled data sets. Note, that current state of art is that a data scientist needs to be involved to customize a similarity measure to reflect the expectations of an end-user. The data scientist is often used to determine what features are relevant to a specific outcome. For example for cost information, the data scientist would determine what features found in the data affect cost and then use the identified features in a clustering algorithm. It is hard to identify data features that affect meaningful grouping in unlabeled data.

The embodiments described herein provide an automated way of choosing an appropriate similarity measure to be used in clustering based upon information that the end-users know. Hence, it allows for making available clustering techniques to end-users that do not have a data analytics background. In particular, it allows doctors, quality managers, CEO's, and other administrators to use these techniques for e.g., population health management.

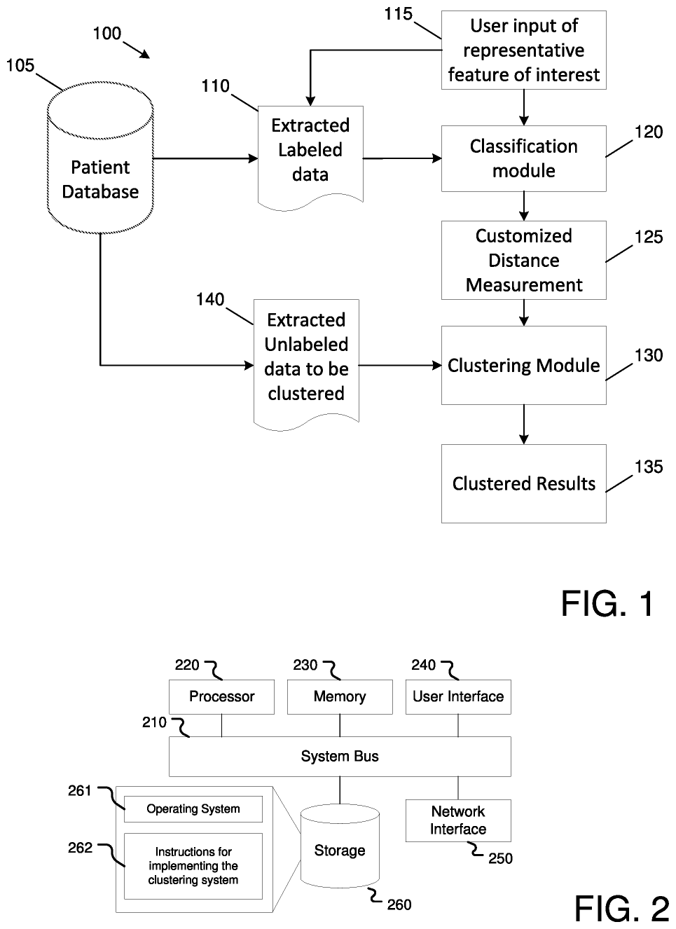

FIG. 1 illustrates a block diagram for a user defined transferable clustering system. The clustering system includes a patient database 105 that includes electronic health records (EHR) for patients. This database may include the EHR for a specific medical practice, medical facility, or medical system. A user inputs a representative feature of interest 115. In the case of a categorical feature, such a feature may be used directly. In the case of a continuous feature, a median split can be performed to create binary labels. Alternatively, depending on the distribution of the feature, a different categorization can be performed based upon ranges of the continuous feature. An example of such a feature may be overall cost for heart bypass surgery. The user may provide as set of cost thresholds, for example $30K and 50K, to provide three different cost groupings (i.e., <$30, $30K to $50K, and >$50K). If the average cost of heart bypass surgery is $40K, then such labels help to group patients into situations that fall within +/−$10K of the average cost, or above or below this range. Such an understanding would help an administrator identify patients that might lead to higher or lower costs than normal. This representative feature of interest and the users definition of labels are then used to extract labeled data 110 from the patient database 105. In the heart bypass surgery example, all data for patients who have undergone heart surgery with available cost data is extracted from the patient database 105. Then a cost label is placed on the extracted data.

The classification module 120 receives the user input of representative feature of interest 115 and the extracted labeled data 110. The classification module then trains a classifier to predict these labels and to produce a customized distance measure. The classification technique should be one that combines the task of classifying with finding an optimized data transformation that reflects the classification task. Such a classifier will transform the input data to a data space that causes data similar to the labeled data to be grouped closer together and farther from data in the other groups. Examples of these techniques are logistic regression (where the regression-weights perform an optimized linear transformation to a single dimension) or Generalized Learning Vector Quantization (GLVQ/GMLVQ; where a weighted distance measure is optimized and performs a linear mapping of the data). Any other metric learning method may be used. See for example Juan Luis Suarez, Salvador Garcia, Francisco Herrera, A Tutorial on Distance Metric Learning: Mathematical Foundations, Algorithms and Experiments, arXiv:1812.05944v2. The classification module 120 produces the customized distance measurement 125.

The customized distance measurement 125 may be used to transform unlabeled data into a space that tells a user something about the labels that were used to train the customized distance measurement. Once the data has been transformed into the new data space clustering of the data will be effective. The dimensionality of the new space may be the same or less than the dimensionality of the original data. Further, the customized distance measurement will use weights that weigh the contribution of each feature in the input data to the output data. As different features of interest are used, these weights will change accordingly.

Now with the customized distance measurement 125 the user may now seek to cluster unlabeled patient data. The clustering module 130 extracts unlabeled data to be clustered 140 from the patient database. Such data may be selected based upon various criteria of interest to the user of the system. In some situations the unlabeled data may not have all of the data features used by the customized distance measure. In such situations, data imputation techniques may be used to estimate a value for the missing data elements.

The clustering module 130 then applies a clustering technique on the extracted unlabeled data using the customized distance measurement to produce clustered results 135. These clustered results cluster the patients in the extracted data to produce clusters corresponding to the labels identified by the user. A common clustering technique is k-means. Other clustering techniques that may be used include: Hierarchical Bottom-up/Top-down connectivity based methods such as Agglomerative Hierarchical Clustering/Single Linkage, Minimum Spanning Tree methods, or Divisive; Centroid-based methods including K-Means/Medians/Modes; Prototype based methods including Vector Quantization and Neural Gas; Distribution/Density based methods such as DBSCAN and OPTICS; and Fuzzy variants methods such as Fuzzy c-means.

Using the bypass heart surgery example, it is noted that often the costs associated with a patients treatment lags behind the other data in the patient database. Accordingly, a hospital administrator may use the customized distance measurement along with the clustering module to classify current patients costs which may then be used for budgeting purposes.

From a technical perspective, by using labelled data a mapping of the data to a space that reflects the separation in terms of the labels is created. This mapping is applied to a new dataset to also reflect that separation in the new dataset. This obviates the need to create such mapping on the new dataset itself, which is often impossible due to the target-dataset having no labels.

In this way, the knowledge of how to optimally transform the data (i.e., based upon using labeled data) to represent differences with respect to a feature of interest is leveraged to then be used in the clustering module which will, thereby, also reflect differences that align (but are not limited to) with the feature of interest.

Note that the creation of the customized distance measure may be done on a different dataset than the application of the clustering as long as the datasets are not too dissimilar from one-another. For example, within a consortium of hospitals in geographic region or country, one could train the similarity measure on the population of one hospital and apply it to clustering the data of other hospitals within the consortium. In another example the hospital population of 2015-2017 may be used to train the customize distance measure, which them may be applied to cluster the hospital population of 2018-2019.

The clustering system 100 may be used by a variety of different users to extract meaningful grouping form the same set of unlabeled data based upon the users input of a representative feature of interest. As an example, consider a CEO (as end-user) who wants to identify subgroups of his patient population that show some differences in healthcare finances. For that reason, he selects “total yearly cost of care” as a feature of interest that is used by the classification module and trains a customized similarity measure using his patient population of 2015-2017. The customized similarity measure will now reflect differences in total yearly cost of care (but also other features that are correlated).

Now he applies the clustering method to his more recent population (2018-2019) for which the financial data is not yet up to date (and thus cannot be used as label) and patients are differentiated based upon the similarity measure that reflects aspects of their healthcare costs. Groups of high cost versus groups of low costs are found, and given that, e.g., there is an effect of age on the costs (reflected in the data), the subgroups will also reflect differences in age.

In contrast, consider a care manager using the same data, who is more interested in observing differences in clinical state of the patient. The care manager selects “cholesterol level” as feature of interest and finds with the clustering subgroups that go more with high vs low cholesterol level, but also finds that subgroups are differentiated based upon lifestyle parameters that are correlated with cholesterol level.

Hence, this method allows for steering the results of data driven and unsupervised analysis to better reflect effects of interest from the end-users.

FIG. 2 illustrates an exemplary hardware diagram 200 for implementing the user defined transferable clustering system of FIG. 1. As shown, the device 200 includes a processor 220, memory 230, user interface 240, network interface 250, and storage 260 interconnected via one or more system buses 210. It will be understood that FIG. 2 constitutes, in some respects, an abstraction and that the actual organization of the components of the device 200 may be more complex than illustrated.

The processor 220 may be any hardware device capable of executing instructions stored in memory 230 or storage 260 or otherwise processing data. As such, the processor may include a microprocessor, a graphics processing unit (GPU), field programmable gate array (FPGA), application-specific integrated circuit (ASIC), any processor capable of parallel computing, or other similar devices.

The memory 230 may include various memories such as, for example L1, L2, or L3 cache or system memory. As such, the memory 230 may include static random-access memory (SRAM), dynamic RAM (DRAM), flash memory, read only memory (ROM), or other similar memory devices.

The user interface 240 may include one or more devices for enabling communication with a user and may present information to users. For example, a user of the clustering system may enter information regarding features of interest, and then the clustering results may be presented to the user on user interface 240. For example, the user interface 240 may include a display, a touch interface, a mouse, and/or a keyboard for receiving user commands. In some embodiments, the user interface 240 may include a command line interface or graphical user interface that may be presented to a remote terminal via the network interface 250. The user interface 240 may be used to display the graphical performance display.

The network interface 250 may include one or more devices for enabling communication with other hardware devices. For example, the network interface 250 may include a network interface card (NIC) configured to communicate according to the Ethernet protocol or other communications protocols, including wireless protocols. Additionally, the network interface 250 may implement a TCP/IP stack for communication according to the TCP/IP protocols. Various alternative or additional hardware or configurations for the network interface 250 will be apparent.

The storage 260 may include one or more machine-readable storage media such as read-only memory (ROM), random-access memory (RAM), magnetic disk storage media, optical storage media, flash-memory devices, or similar storage media. In various embodiments, the storage 260 may store instructions for execution by the processor 220 or data upon with the processor 220 may operate. For example, the storage 260 may store a base operating system 261 for controlling various basic operations of the hardware 200. The storage 262 may store instructions for implementing the clustering system described above. Further, the storage 260 may implement the patient database 105.

It will be apparent that various information described as stored in the storage 260 may be additionally or alternatively stored in the memory 230. In this respect, the memory 230 may also be considered to constitute a “storage device” and the storage 260 may be considered a “memory.” Various other arrangements will be apparent. Further, the memory 230 and storage 260 may both be considered to be “non-transitory machine-readable media.” As used herein, the term “non-transitory” will be understood to exclude transitory signals but to include all forms of storage, including both volatile and non-volatile memories.

While the system 200 is shown as including one of each described component, the various components may be duplicated in various embodiments. For example, the processor 220 may include multiple microprocessors that are configured to independently execute the methods described herein or are configured to perform steps or subroutines of the methods described herein such that the multiple processors cooperate to achieve the functionality described herein. Such plurality of processors may be of the same or different types. Further, where the device 200 is implemented in a cloud computing system, the various hardware components may belong to separate physical systems. For example, the processor 220 may include a first processor in a first server and a second processor in a second server.

The clustering system described herein provides a technological improvement over current medical data clustering systems. The clustering system allows a user to specify parameters or features of interest, and this may be used to extract data from the patient database to train a customized distance measurement. This may then be used to cluster patient data of interest based upon the user specified features. In the past a data scientist has to be employed to identify features of interest to cluster unlabeled data according to the desired clustering of a user. The disclosed clustering system allows a user to specify the features and labels of interest and then a customized distance measurement is generated and used to cluster unlabeled data. Further, this customized distance measurement may be used on other patient databases, different from the data used to train the distance measure. Then another user may specify a different feature or parameter of interest and use the same system and data to develop a different customized distance measure that may be used to cluster unlabeled data. This clustering system provides a tool to allow a user to cluster together patients according to a user specified feature of interest.

Any combination of specific software running on a processor to implement the embodiments of the invention, constitute a specific dedicated machine.

As used herein, the term “non-transitory machine-readable storage medium” will be understood to exclude a transitory propagation signal but to include all forms of volatile and non-volatile memory.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1. A method for clustering patients based upon unlabeled patient medical data, comprising:
receiving a first feature of interest from a first user;
extracting first patient data from a first patient database based upon the first feature of interest;
labeling the extracted first patient data based upon the first feature of interest;
producing a first customized distance measure using a classifier on the labeled patient data;
extracting first unlabeled patient data from a second patient database; and
clustering the first unlabeled patient data using a clustering technique and the first customized distance measure to produce first clustered results.

2. The method of claim 1, wherein the second patient database is the same as the first patient database.

3. The method of claim 1, further comprising:
receiving a second feature of interest from a second user;
extracting second patient data from a second patient database based upon the second feature of interest;
labeling the extracted second patient data based upon the second feature of interest;
producing a second customized distance measure using a classifier on the second labeled patient data; and
clustering the second unlabeled patient data using a clustering technique and the second customized distance measure to produce second clustered results.

4. The method of claim 1, wherein the feature of interest is a continuous value.

5. The method of claim 1, wherein the feature of interest is categorical.

6. The method of claim 1, wherein the feature of interest is a binary value.

7. A non-transitory machine-readable storage medium encoded with instructions for clustering patients based upon unlabeled patient medical data, comprising:
instructions for receiving a first feature of interest from a first user;
instructions for extracting first patient data from a first patient database based upon the first feature of interest;
instructions for labeling the extracted first patient data based upon the first feature of interest;
instructions for producing a first customized distance measure using a classifier on the labeled patient data;
instructions for extracting first unlabeled patient data from a second patient database; and
instructions for clustering the first unlabeled patient data using a clustering technique and the first customized distance measure to produce first clustered results.

8. The non-transitory machine-readable storage medium of claim 7, wherein the second patient database is the same as the first patient database.

9. The non-transitory machine-readable storage medium of claim 7, further comprising:
instructions for receiving a second feature of interest from a second user;
instructions for extracting second patient data from a second patient database based upon the second feature of interest;
instructions for labeling the extracted second patient data based upon the second feature of interest;
instructions for producing a second customized distance measure using a classifier on the second labeled patient data; and
instructions for clustering the second unlabeled patient data using a clustering technique and the second customized distance measure to produce second clustered results.

10. The non-transitory machine-readable storage medium of claim 7, wherein the feature of interest is a continuous value.

11. The non-transitory machine-readable storage medium of claim 7, wherein the feature of interest is categorical.

12. The non-transitory machine-readable storage medium of claim 7, wherein the feature of interest is a binary value.

13. A device, for clustering patients based upon unlabeled patient medical data comprising:
a memory;
a processor coupled to the memory, wherein the processor is further configured to:
receive a first feature of interest from a first user;
extract first patient data from a first patient database based upon the first feature of interest;
label the extracted first patient data based upon the first feature of interest;
producing a first customized distance measure using a classifier on the labeled patient data;
extract first unlabeled patient data from a second patient database; and
cluster the first unlabeled patient data using a clustering technique and the first customized distance measure to produce first clustered results.

14. The device of claim 13, wherein the second patient database is the same as the first patient database.

15. The device of claim 13, wherein the process is further configured to:
receive a second feature of interest from a second user;
extract second patient data from a second patient database based upon the second feature of interest;
label the extracted second patient data based upon the second feature of interest;
produce a second customized distance measure using a classifier on the second labeled patient data; and
cluster the second unlabeled patient data using a clustering technique and the second customized distance measure to produce second clustered results.

16. The device of claim 13, wherein the feature of interest is a continuous value.

17. The device of claim 13, wherein the feature of interest is categorical.

18. The device of claim 13, wherein the feature of interest is a binary value.
